(19) United States
(12) Patent Application Publication
(10) Pub. No.: US 2010/0286226 A1
(43) Pub. Date: Nov. 11, 2010

(54) COMBINATION THERAPY RELATED TO SEROTONIN DUAL ACTION COMPOUNDS

(75) Inventors: Connie Sanchez Morillo, West Milford, NJ (US); Toni D. Wolinsky, Haworth, NJ (US)

Correspondence Address: LUNDBECK RESEARCH USA, INC.;ATTENTION: STEPHEN G. KALINCHAK, LEGAL, 215 COLLEGE ROAD, PARAMUS, NJ, 07652, US

(73) Assignee: H. LUNDBECK A/S, Valby-Copenhagen (DK)

(21) Appl. No.: 12/679,789

(22) PCT Filed: Sep. 24, 2008
(86) PCT No.: PCT/US2008/077435

§ 371 Date: Jul. 23, 2010

Related U.S. Application Data
(60) Provisional application No. 60/974,595, filed on Sep. 24, 2007.

(51) Int. Cl. A61K 31/40; A61P 25/24; A61P 25/22; A61P 3/04; A61P 25/30; A61P 25/00

(52) U.S. Cl. 514/418

(57) ABSTRACT

The present invention relates to combination therapies and pharmaceutical compositions comprising a combination of 5-hydroxytryptophan and a serotonin reuptake inhibitor that binds to an ailosteric site of the serotonin transporter. The present invention further provides a pharmaceutical composition comprising (i) 5-hydroxytryptophan in an amount ranging from about 1 mg to about 75 mg; and (ii) a serotonin reuptake inhibitor that binds to an allosteric site of the serotonin transporter.

### FIELD OF INVENTION

The present invention relates to combination therapies and pharmaceutical compositions with improved efficacy comprising a combination of 5-hydroxytryptophan and a serotonin reuptake inhibitor which binds to an allosteric site of the serotonin transporter.

### BACKGROUND OF THE INVENTION

Throughout this application, various publications are referenced. The disclosure of these publications in their entireties are hereby incorporated by reference into this application to describe more fully the art to which this invention pertains.

5-hydroxytryptophan (5-HTP) is the direct precursor to serotonin (5-hydroxytryptamine; 5-HT). In vivo, 5-HTP is decarboxylated to produce 5-HT. 5-HT levels in the brain are dependent on levels of 5-HTP in the central nervous system (CNS). No transport molecules are necessary to transport 5-HTP across the blood-brain barrier. 5-HTP has been clinically shown to increase production of serotonin in the brain and therefore 5-HTP administration has been suggested as a treatment for patients with mild or moderate depression (for review, see Meyers, S., Altern Med Rev. 2000 February, 5(1):64-71; and Birdsall, T. C., Altern Med Rev. 1998 August; 3(4):271-80).

Serotonin reuptake inhibitors (SRIs) have become first choice therapeutics in the treatment of affective disorders, especially depression, because they are effective, well tolerated and have a favorable safety profile compared to the classic tricyclic antidepressants.

However, there is virtually no pharmaceutical treatment known that does not, apart from its benefits to patients, also carry some degree of risk of adverse side effects. 5-HTP monotherapy has been associated with gastrointestinal (nausea, vomiting, diarrhea) and psychopathological (acute anxiety state, hypomania) side effects in open studies with human patients (Zmilacher, K., Battegay, R. and Gastpar, M., Neuropsychobiology. 1988, 20(1):28-35; Gijsman, H. J., et al., J Clin Psychopharmacol. 2002 April, 22(2):183-9). 5-HTP administration has been implicated as a possible cause of Eosinophilia-Myalgia Syndrome (for review, see Das. Y. T., et al., Toxicol Lett. 2004 Apr. 15; 150(1):111-22.). One approach to managing these risks of side effects may be to lower the dose of 5-HTP.

With respect to SRIs, possible side effects to be balanced against the known benefits of SRIs and to be managed may include sexual dysfunction and sleep disturbances. Many patients experience delayed onset of a therapeutic effect during SRI monotherapy. Further clinical studies on depression and anxiety disorders indicate that more than 30% of patients treated with SRI monotherapy as a class are non-responsive.

Observations about the varying potentiation effects of different SRIs when administered with 5-HTP in various animal models have been noted. For example, Sanchez, C. and Hyttel, J., European Journal of Pharmacology (1994) 264:241-247 observed that a subeffective dose of L, 5-HTP greatly potentiated the antiaggressive effect of citalopram and paroxetine in an isolation-induced aggression mouse model.

C. Sanchez, European Journal of Pharmacology (2003) 464:155-158, also tested co-administration of L, 5-HTP with citalopram or escitalopram in an ultrasonic vocalization rat model for anxiety. In that model, in which ultrasonic vocalization is theorized to mimic panic anxiety in the rat, it was observed that the anxiolytic response to co-treatment of L, 5-HTP with citalopram was slightly attenuated and co-treatment of L, 5-HTP with escitalopram was markedly enhanced. Concomitant treatment with R-citalopram produced a significant increase of ultrasonic vocalization compared to controls.

Thus, patients may benefit from administration of a lower dose of 5-HTP. Patients may also benefit from administration of a lower dose of an SRI. Furthermore, patients that do not respond to SRIs may benefit from a combination therapy of an SRI and 5-HTP. Such combination therapy includes lower doses of either SRI or 5-HTP, yet may achieve greater efficacy or earlier onset of therapeutic effect than with SRI or 5-HTP monotherapy. Such combination therapy is particularly beneficial when the SRI binds to an allosteric site of the serotonin transporter (SERT).

### SUMMARY OF THE INVENTION

An objective of the present invention is to provide a pharmaceutical composition comprising (i) 5-hydroxytrytophan and (ii) 2-(6-Fluoro-1H-indol-3-ylsulfanyl)-benzyl]-methyl-amine.

Another objective of the present invention is to provide a pharmaceutical composition comprising (i) 5-hydroxytryptophan in an amount ranging from about 1 mg to about 75 mg; and (ii) a serotonin reuptake inhibitor that binds to an allosteric site of the serotonin transporter; 2-(6-Fluoro-1H-indol-3-ylsulfanyl)-benzyl]-methyl-amine.

### BRIEF DESCRIPTION OF THE FIGURES

FIG. 1. Firing rate histogram representative of a spontaneously firing serotonin-containing neuron in the dorsal raphe of an anesthetized rat. Injection of a low threshold dose of 5-HTP (25 mg/kg) (left arrow) reduces cell firing rate. A further decrease is observed following a second injection of 25 μg/kg of Compound II, 2-(6-Fluoro-1H-indol-3-ylsulfanyl)-benzyl]-methyl-amine (right arrow).

FIG. 2. Firing rate histogram representative of a spontaneously firing serotonin-containing neuron in the dorsal raphe of an anesthetized rat. Compound II, 2-(6-Fluoro-1H-indol-3-ylsulfanyl)-benzyl]-methyl-amine (left arrow), is administered prior to 25 mg/kg 5-HTP (middle arrow) in experiments similar to those presented in FIG. 1. The second injection (5-HTP) greatly decreased cell firing. The neuron responds to subsequent administration of WAY-100635 (a selective 5-HT_{1A} antagonist) (right arrow) which increases the spontaneous firing rate of the neuron.

FIG. 3. Graphical summary of the combination dosing of Compound II and 5-HTP on dorsal raphe cell firing. Regardless of the treatment order, the combination of this SRI and this 5-HT precursor produces an effect on neuronal activity that is greater than that achieved by simply adding the individual dose effects. The combination of 5-HTP injected after Compound II has a significantly greater effect on cell firing rate than 5-HTP alone (**p<0.01). Similarly, the effect of 5-HTP followed by injection of Compound II on cell firing rate is significantly greater than the effect of Compound II alone (**p<0.01). Statistical analysis was performed on data collected from six different rats.

### DETAILED DESCRIPTION OF THE INVENTION

The present invention relates to a pharmaceutical composition comprising 5-hydroxytryptophan and a serotonin reuptake inhibitor that binds to an allosteric site of the serotonin transporter, for example 2-(6-Fluoro-1H-indol-3-ylsulfanyl)-benzyl]-methyl-amine.

As used herein, “serotonin reuptake inhibitor” or “SRI” means a compound which primarily or partly exerts its therapeutic effect by binding to the primary ligand binding site of the serotonin transporter to inhibit serotonin reuptake in the central nervous system (CNS).

As used herein, “allosteric modulator” shall mean an SRI that binds to an allosteric site of the serotonin transporter (SERT). Such compounds are also called allosteric serotonin reuptake inhibitors (ASRIs).

In one embodiment of the invention, the allosteric modulator binds to one or more allosteric sites of the SERT. In another embodiment, the allosteric modulator binds to the allosteric site of the SERT capable of binding 2-(6-Fluoro-1H-indol-3-ylsulfanyl)-benzyl]-methyl-amine.

5-hydroxytryptophan (5-HTP) is an aromatic amino acid naturally produced in the body from amino acid L-tryptophan. 5-HTP is the direct precursor to 5-HT. The formula of 5-HTP is shown below as Formula I.

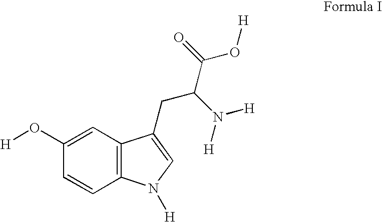

5-HTP is also known as 2-amino-3-(5-hydroxy-1H-indol-3-yl)-propanoic acid (C₁₁H₁₂N₂O₃). Throughout the description and the claims, “5-HTP” and “5-hydroxytrytophan” are intended to include any form of the amino acid 5-hydroxytryptophan, including the base (zwitter ion), pharmaceutically acceptable salts, hydrates or solvates of the base or salt, as well as anhydrates, and also amorphous, or crystalline forms. As used herein, “pharmaceutically acceptable salts” includes salts with pharmaceutically acceptable acids or bases. With respect to 5-HTP, such salts may be formed with pharmaceutically acceptable bases, particularly strong bases such as sodium potassium or ammonium hydroxide. Such salts of 5-HTP may also be formed with pharmaceutically acceptable acids, such as hydrochloric acid, hydrobromic acid, phosphoric acid, maleic acid, oxalic acid, tartaric acid and the like. Accordingly, 5-HTP may be used in the form of an acid addition salt, or in the form of a zwitter ion hydrate, zwitter ion monohydrate, or zwitter ion anhydrite.

For the purposes of this invention, 5-HTP may be in a racemic mixture or as the substantially pure D-enantiomer, D-5-hydroxytryptophan, or as the substantially pure L-enantiomer, L-5-hydroxytryptophan.

One aspect of the present invention relates to a pharmaceutical composition comprising 5-HTP for use in a combination therapy with an SRI.

Another aspect of the present invention provides a pharmaceutical composition comprising (i) 5-hydroxytryptophan in an amount ranging from about 1 mg to about 75 mg; and (ii) 2-(6-Fluoro-1H-indol-3-ylsulfanyl)-benzyl]-methyl-amine.

In accordance with the present invention described herein, 5-HTP may be used to augment and/or provide an earlier onset of the therapeutic effect of serotonin reuptake inhibitors, such as allosteric modulators. Further, as part of the present invention, lower doses of 5-HTP when used in combination therapy may augment and/or provide an earlier onset of the therapeutic effect of an allosteric modulator. In one embodiment of the invention, 5-HTP in an amount ranging from about 1 mg to about 75 mg is coadministered with 2-(6-Fluoro-1H-indol-3-ylsulfanyl)-benzyl]-methyl-amine. In another embodiment of the invention, 5-HTP in an amount ranging from about 3 mg to about 50 mg is coadministered with 2-(6-Fluoro-1H-indol-3-ylsulfanyl)-benzyl]-methyl-amine. In still another embodiment of the invention, 5-HTP in an amount ranging from about 10 mg to about 50 mg is coadministered with 2-(6-Fluoro-1H-indol-3-ylsulfanyl)-benzyl]-methyl-amine.

As used herein, “augmenting” shall mean improving the therapeutic effect and/or potentiating the effect of an SRI.

As described herein, 2-(6-Fluoro-1H-indol-3-ylsulfanyl)-benzyl]-methyl-amine is an SRI which forms an embodiment of the present invention, or a pharmaceutically acceptable salt of any of these compounds. The formula of 2-(6-Fluoro-1H-indol-3-ylsulfanyl)-benzyl]-methyl-amine is shown as Formula II.

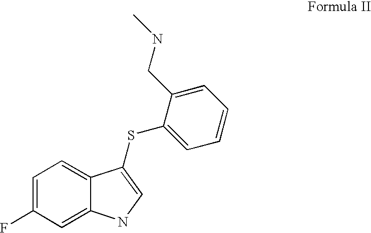

The compound of Formula II, hereinafter referred to as Compound II, exerts an inhibitory effect at both the serotonin transporter (SERT) and the norepinephrine transporter (NET), as measured by in vitro reuptake inhibitory potency, and, as such, is considered a serotonin-norepinephrine reuptake inhibitor (SNRI). As used herein, norepinephrine also means noradrenaline.

Thus not wishing to be bound by a particular theory, Applicants have observed that Compound II meets the definitions of an SRI, an SNRI, and an ASRI.

Compound II mentioned above may be used in the form of the free base or in the form of a pharmaceutically acceptable salt, such as an acid addition salt, the latter being obtainable by a reaction of the base form with an appropriate acid.

In other words, in one embodiment of the invention, 2-(6-Fluoro-1H-indol-3-ylsulfanyl)-benzyl]-methyl-amine, is in the form of pharmaceutically acceptable salt.

For example, 2-(6-Fluoro-1H-indol-3-ylsulfanyl)-benzyl]-methyl-amine may be used in the form of the L-(+)-hydrogen tartrate salt.

In another embodiment, 2-(6-Fluoro-1H-indol-3-ylsulfanyl)-benzyl]-methyl-amine is not the free base in a non-crystalline form.

In other embodiments, such salts may include pharmaceutical acceptable acid addition salts, pharmaceutically acceptable metal salts, ammonium and alkylated ammonium salts. Acid addition salts include salts of inorganic acids as well as organic acids. Also intended as pharmaceutical acceptable acid addition salts are the hydrates, which the present compound, is able to form. Examples of suitable inorganic acids and organic acids are described in WO05/061455A1, which is hereby incorporated by reference in its entirety.

Antidepressant compounds that demonstrate potent inhibition of serotonin reuptake (e.g. SRIs) also inhibit dorsal raphe neuronal firing rates (Rigdon, G C and Wang, C M, Drug Development Research 1991, 22:135-140). As such, co-administration of 5-HTP and an SRI, namely Compound II, or a pharmaceutically acceptable salt thereof, is shown herein to have a greater effect on dorsal raphe neuronal firing than the administration of either compound alone. The surprising effects of combined administration of 5-HTP and this SRI are determined to be synergistic, and not additive, thereby providing improved therapeutic potential.

As mentioned above, in one embodiment of the invention, lower doses of 5-HTP than normally used in monotherapy may be used in combination with a dose of Compound II normally used in monotherapy to augment the 5-HT output and thereby may provide an earlier onset of the therapeutic effect.

In some embodiments, the amount of 5-HTP to be used in combination therapy may range from about 1 to about 75 mg per day, such as from about 3 to about 50 mg per day, or from about 10 to about 50 mg per day. Pharmaceutical compositions of the present invention may therefore comprise from about 1 to about 75 mg, such as from about 3 to about 50 mg, or from about 10 to about 50 mg 5-HTP.

As used herein, a “therapeutically effective amount” of a compound means an amount of a compound sufficient to cure, alleviate or partially arrest the clinical manifestations of a given disease and its symptoms and/or complications. An amount adequate to accomplish this is defined as “therapeutically effective amount”. Effective amounts for each purpose will depend on the severity of the disease or injury as well as the weight and general state of the subject. It is understood that determining an appropriate dosage may be achieved using routine experimentation, by constructing a matrix of values and testing different points in the matrix, which is all within the ordinary skills of a trained physician.

In one embodiment of the invention, the pharmaceutical composition comprises a therapeutically effective amount of 2-(6-Fluoro-1H-indol-3-ylsulfanyl)-benzyl]-methyl-amine. In a further embodiment, the pharmaceutical composition comprises from 0.1 mg to 50 mg of 2-(6-Fluoro-1H-indol-3-ylsulfanyl)-benzyl]-methyl-amine. Also included in the present invention is the administration of such pharmaceutical composition to a patient in need thereof, so that the daily dose ranges of 2-(6-Fluoro-1H-indol-3-ylsulfanyl)-benzyl]-methyl-amine are 0.1 mg to 50 mg per day.

As used herein, “subeffective dose” shall mean a dose in an amount less than the lowest dose that is administered to achieve a clinical result as a monotherapy.

In yet another aspect of the invention, the pharmaceutical composition comprises 2-(6-Fluoro-1H-indol-3-ylsulfanyl)-benzyl]-methyl-amine in an amount less than 50 mg and 5-HTP in an amount ranging from about 1 mg to about 600 mg. In another embodiment of the invention, the pharmaceutical composition comprises 2-(6-Fluoro-1H-indol-3-ylsulfanyl)-benzyl]-methyl-amine in an amount less than 50 mg and 5-HTP in an amount ranging from about 25 mg to about 300 mg.

In still another embodiment of the invention, the pharmaceutical composition comprises 2-(6-Fluoro-1H-indol-3-ylsulfanyl)-benzyl]-methyl-amine in an amount less than 50 mg and 5-HTP in an amount ranging from about 50 mg to about 200 mg. In a further embodiment of the invention, the pharmaceutical composition comprises 2-(6-Fluoro-1H-indol-3-ylsulfanyl)-benzyl]-methyl-amine in an amount from about 0.1 mg to about 49.9 mg.

In further embodiments of the invention, the amount of 5-HTP to be used in combination therapy may range from about 1 mg to about 600 mg per day, such as from about 25 mg to about 300 mg per day, or from about 50 mg to about 200 mg per day. Pharmaceutical compositions of the present invention may therefore comprise from about 1 mg to about 600 mg, such as from about 25 mg to about 300 mg, or from about 50 mg to about 200 mg 5-HTP.

Accordingly, one embodiment of the present invention includes a pharmaceutical composition comprising a subeffective dose of Compound II and 5-HTP, wherein the composition comprises 5-HTP in an amount ranging from about 1 mg to about 600 mg, from about 25 mg to about 300 mg, or from about 50 mg to about 200 mg. Also included in the present invention is the administration of such pharmaceutical compositions to a patient in need thereof, so that the daily dose ranges of 5-HTP are from about 1 mg to about 600 mg per day, or about 25 mg to about 300 mg per day, or about 50 mg to 200 mg per day

Aromatic amino acid decarboxylases that degrade 5-HTP to serotonin are widely distributed throughout the body. A peripheral decarboxylation inhibitor can be administered in combination with 5-HTP to prevent the degradation of 5-HTP to serotonin.

Thus, the pharmaceutical composition may further comprise a peripheral decarboxylation inhibitor. Peripheral decarboxylation inhibitors include, but are not limited to, carbidopa, L-α-methyldopa, monofluoromethyldopa, difluoromethyldopa and benserazide.

Pharmaceutical compositions of the present invention may contain carbidopa in an amount ranging from about 100 mg to about 150 mg.

According to the invention, the pharmaceutical compositions described herein may be administered in any suitable way, e.g. orally or parentally, and it may be presented in any suitable form for such administration, e.g. in the form of tablets, capsules, powders, syrups or solutions or dispersions for injection. In one embodiment of the present invention, the composition is administered in the form of a solid pharmaceutical entity, suitably as a tablet or a capsule or in the form of a suspension, solution or dispersion for injection.

Methods for the preparation of solid pharmaceutical compositions are well known in the art. For example, tablets may thus be prepared by mixing the active ingredients with ordinary adjuvants and/or diluents and subsequently compressing the mixture in a convenient tabletting machine. Examples of adjuvants or diluents comprise: corn starch, lactose, talcum, magnesium stearate, gelatin, gums, and the like. Other adjuvants or additives such as colorings, aroma, preservatives, etc. may also be used provided that they are compatible with the active ingredients.

The pharmaceutical compositions can be administered as part of the claimed invention as an oral dose form, such as a solid dose form, typically tablets or capsules, or as a liquid oral dose form. The pharmaceutical compositions described herein are most conveniently administered in unit dosage forms such as tablets or capsules. For example, such tablets or capsules may contain 5-HTP in amounts ranging from about 1 to about 600 mg, or from about 25 mg to about 300 mg, or from about 10 to 50 mg.

To prepare the pharmaceutical composition of this invention, an appropriate amount of 5-HTP and/or Compound II, in salt form or base form, is combined in an intimate admixture with a pharmaceutically acceptable carrier, which can take a wide variety of forms depending on the form desired for administration. Those pharmaceutical compositions may be in unitary dosage form suitable for administration orally, rectally, percutaneously, or by parenteral injection. For example, in preparing the compositions in oral dosage form, any of the usual pharmaceutical media, such as water, glycols, oils, alcohols, and the like, may be incorporated in the form of oral liquid preparations. Oral liquid preparations may be suspensions, syrups, elixirs, and solutions. In preparing the compositions in oral dosage form, any of the usual pharmaceutical media, such as starches, sugars, kaolin, lubricants, binders, disintegrating agents, and the like, may be incorporated in the form of solid carriers. Oral solid preparations may be powders, pills, capsules and tablets. Because of their ease in administration, tablets and capsules represent the most advantageous oral dosage form, in which case solid pharmaceutical carriers would be employed.

It is especially advantageous to formulate the aforementioned pharmaceutical compositions in a unitary dosage form for ease of administration and uniformity of dosage. As used herein, unitary dosage form means physically discrete units suitable as unitary dosages, each unit containing a predetermined quantity of 5-HTP and/or Compound II calculated to produce the desired therapeutic effect, in association with the required pharmaceutical carrier. Examples of unitary dosage forms are tablets (including scored coated tablets), capsules, pills, powder packets, wafers, injectable solutions or suspensions, and the like, and combinations thereof.

5-HTP may be administered before, during or after the administration of Compound II, provided that the time between administration of 5-HTP and the administration of Compound II is such that ingredients are allowed to act synergistically on the central nervous system. When simultaneous administration of 5-HTP and Compound II is envisaged, a single composition containing both Compound II and 5-HTP may be particularly convenient. Alternatively, the serotonin reuptake inhibitor and 5-HTP may be administered separately in the form of suitable compositions. Such pharmaceutical compositions may further comprise a peripheral decarboxylation inhibitor. The compositions may be prepared as described hereinabove. Thus, such compositions may comprise Compound II and a peripheral decarboxylation inhibitor, such as carbidopa. Other compositions may comprise 5-HTP and a peripheral decarboxylation inhibitor, such as carbidopa. Such compositions may be administered simultaneously, such as in a single tablet, and the like, or may be administered separately, such as in separate compositions or tablets, and the like.

The present invention also comprises 5-HTP and Compound II, as a combination preparation for simultaneous, separate or sequential use in psychiatric drug therapy. Such compositions may comprise, for example, a kit comprising discrete unit dosage forms containing 5-HTP and discrete unit dosage forms of Compound II, all contained in the same container or pack, e.g. a blister pack. Such pharmaceutical compositions may further comprise a peripheral decarboxylation inhibitor. The above mentioned compositions are made in accord with any aspects of the present invention described herein.

In some embodiments, the invention relates to a kit comprising a dose of 2-(6-Fluoro-1H-indol-3-ylsulfanyl)-benzyl]-methyl-amine and 5-HTP. In some embodiments, the invention relates to a kit comprising a dose of 2-(6-Fluoro-1H-indol-3-ylsulfanyl)-benzyl]-methyl-amine and 5-HTP in an amount ranging from about 1 mg to about 600 mg, in an amount ranging from about 25 mg to about 300 mg or in an amount ranging from about 50 mg to about 200 mg.

In some embodiments, the invention relates to a kit comprising 2-(6-Fluoro-1H-indol-3-ylsulfanyl)-benzyl]-methyl-amine, and 5-HTP in an amount ranging from about 1 mg to about 75 mg, in an amount ranging from about 3 mg to about 50 mg or in an amount ranging from about 10 mg to about 50 mg. In further embodiments, the kit comprises 2-(6-Fluoro-1H-indol-3-ylsulfanyl)-benzyl]-methyl-amine in an amount ranging from about 0.1 mg to about 50 mg.

In other embodiments, the invention relates to a kit comprising 2-(6-Fluoro-1H-indol-3-ylsulfanyl)-benzyl]-methyl-amine and 5-HTP. In some embodiments, the invention relates to a kit comprising 2-(6-Fluoro-1H-indol-3-ylsulfanyl)-benzyl]-methyl-amine and 5-HTP in an amount ranging from about 1 mg to about 600 mg, in an amount ranging from about 25 mg to about 300 mg or in an amount ranging from about 50 mg to about 200 mg. In some aspects, the kit further comprises a peripheral decarboxylation inhibitor.

In other aspects, the invention relates to the pharmaceutical compositions as described herein comprising 5-HTP and 2-(6-Fluoro-1H-indol-3-ylsulfanyl)-benzyl]-methyl-amine for use in combination therapy for the treatment of affective disorders. In another aspect of the invention, the invention relates to the pharmaceutical compositions as described herein comprising 5-HTP and 2-(6-Fluoro-1H-indol-3-ylsulfanyl)-benzyl]-methyl-amine for use in combination therapy for the treatment of depression. In still another aspect, the present invention relates to the pharmaceutical compositions as described herein comprising 5-HTP and 2-(6-Fluoro-1H-indol-3-ylsulfanyl)-benzyl]-methyl-amine for use in combination therapy for the treatment of anxiety disorders.

All of the pharmaceutical compositions described herein may further comprise a peripheral decarboxylation inhibitor.

The invention relates to a method for the treatment of a disease or disorder selected from the group consisting of an affective disorders, such as depression and anxiety disorders including general anxiety disorder, social anxiety disorder, acute stress disorder, post traumatic stress disorder, obsessive compulsive disorder, and panic anxiety in a living animal body, including a human, comprising administering to a subject in need thereof a therapeutically effective amount of Compound II, as the free base or a salt thereof, and 5-HTP.

The invention relates to use of Compound II, as the free base or a salt thereof, for the preparation of a pharmaceutical composition for the treatment of affective disorders, such as depression and anxiety disorders including general anxiety disorder, social anxiety disorder, acute stress disorder, post traumatic stress disorder, obsessive compulsive disorder, and panic anxiety.

In other aspects, the invention relates to the use of 5-HTP for the preparation of a pharmaceutical composition to be used in combination with 2-(6-Fluoro-1H-indol-3-ylsulfanyl)-benzyl]-methyl-amine. In a further aspect, the invention relates to the use of 5-HTP for the preparation of a pharmaceutical composition useful for augmenting and/or providing an earlier onset of the therapeutic effect of 2-(6-Fluoro-1H-indol-3-ylsulfanyl)-benzyl]-methyl-amine.

In still further aspects, the invention relates to a method of treatment of diseases or disorders responsive to an SRI, comprising administering 5-HTP and 2-(6-Fluoro-1H-indol-3-ylsulfanyl)-benzyl]-methyl-amine to a human patient in need thereof.

A further aspect of the invention relates to use of 5-HTP and 2-(6-Fluoro-1H-indol-3-ylsulfanyl)benzyl]-methyl-amine for the preparation of a pharmaceutical composition for the treatment of diseases or disorders responsive to the therapeutic effect of an SRI.

In another aspect, the invention relates to use of 5-HTP for the preparation of a pharmaceutical composition for the treatment of an individual to be treated with or undergoing treatment with an SRI, wherein said individual suffers from diseases or disorders responsive to the therapeutic effect of an SRI. In some aspects, the invention relates to use of 5-HTP for the preparation of a kit for the treatment of an individual to be treated with or undergoing treatment with an SRI, wherein said individual suffers from diseases or disorders responsive to the therapeutic effect of an SRI.

Diseases or disorders responsive to treatment with an SRI include, but are not limited to affective disorders, eating disorders, phobias, dysthymia, premenstrual syndrome, cognitive disorders, impulse control disorders, attention deficit hyperactivity disorder, and drug abuse. Affective disorders include, but are not limited to depression and anxiety disorders. Eating orders include, but are not limited to bulimia, anorexia and obesity. Anxiety disorders include, but are not limited to general anxiety disorder, panic anxiety, obsessive compulsive disorder, acute stress disorder, post trauma stress disorder, and social anxiety disorder.

In other embodiments, the invention relates to a method for augmenting and/or providing an earlier onset of the therapeutic effect of an SRI comprising administering 5-HTP to a human patient o be treated with or undergoing treatment with an SRI.

In another embodiment, the pharmaceutical compositions as described herein are used in the treatment of depression, anxiety disorders and other affective disorders, eating disorders such as bulimia, anorexia and obesity, phobias, dysthymia, premenstrual syndrome, cognitive disorders, impulse control disorders, attention deficit hyperactivity disorder, and drug abuse, in particular depression.

In further embodiments, the pharmaceutical compositions as described herein are used in the treatment of anxiety disorders such as general anxiety disorder, panic anxiety, obsessive compulsive disorder, acute stress disorder, post trauma stress disorder, or social anxiety disorder.

By way of example, the invention will be better understood by the experimental details that follow. One skilled in the art will readily appreciate that the specific methods and results discussed therein are merely illustrative of the invention as described more fully in the claims which follow thereafter.

### EXPERIMENTAL DETAILS

### Mouse Forced Swim Test

Male NMRI/BOM mice (18-25 g; Bomholtgaard, Denmark) may be used. The mice are housed in plastic cages (35×30×12 cm), 10 in each and habituated to the animal facilities for at least a week before test. The room temperature (21+/−2° C.), relative humidity (55+/−5%), and air exchange (16 times per h) are automatically controlled. The animals should have free access to commercial food pellets and tap water before test.

A mouse that is forced to swim in a spatially constrained container will exert a characteristic immobile posture. Pretreatment with an antidepressant will counteract this effect. The test was conducted as described in detail by Sanchez and Meier (Psychopharmacol 129: 197-205; 1997). Briefly, a fully automated test system with 6 swim units (2000 ml glass jars filled with 1200 ml soiled water (23-25° C.) in which a mouse had been placed previously) is used. The assessment of immobility is performed by image analysis.

Thirty minutes after drug or vehicle treatment the mice are treated with 5-HTP and 20 min later the mice are placed into the glass jar and left in the water for a total of 6 min. The accumulated duration of immobility is measured during the last 3 min.

### Rat Microdialysis

Microdialysis in freely moving rats may be performed as described in detail by Mørk, A., Kreilgaard, M. and Sanchez, C. (Neuropharmacology, 2003 August, 45(2):167-73) to study the effect of escitalopram and fluoxetine alone, and in combination with 5-HTP (25 mg/kg, s.c.) an extracellular serotonin levels.

Briefly, male Sprague-Dawley rats are prepared for microdialysis by surgically implanting intracerebral guide cannulas. A microdialysis probe is inserted through the guide cannula. Perfusion of the microdialysis probe with filtered Ringer solution (146 mM NaCl, 3 mM KCl, 1 mM MgCl₂, 1.2 mM CaCl₂) is done before the insertion of the probe and continued for the duration of the experiments at a constant flow of 1 μl/minute into the frontal cortex. After stabilization of the animals, testing is initiated by the injection of the compound of Formula II. A 20 minute sampling regime may be used throughout the experiment. 5-HTP (25 mg/kg, s.c.) is injected 60 minutes following injection of the compound of Formula II. 5-HT levels in the dialysate are measured in each sample by means of HPLC with electrochemical detection.

### Mouse Marble Burying Behavior

Male BALB/cByJ mice (Jackson labs, Bar Harbor, Me.) are housed 5/cage upon arrival, at which time they may be 7-8 weeks of age. Animals are acclimated to the housing facility under standard laboratory conditions for a period of at least one week before testing (lights on at 6:00 AM).

Following a one hour period of acclimation to the test room, animals are dosed with either vehicle (saline) or the compound of Formula II. Thirty minutes later, animals receive an injection of vehicle or 5-HTP (2.5 mg/kg, i.p.). Fifteen minutes after the second injection, animals are individually placed into novel cages in which a layer of Aspen Pine bedding on which two parallel rows of 10 marbles each (i.e. twenty total) are placed. After 30 minutes elapse, the mice are removed from their test cages and returned to their home cages. The number of fully visible marbles (less than ⅔ covered with bedding) are counted and subtracted from 20 to arrive at the number of marbles buried.

### Inhibitory Effect on the Firing Activity of 5-HT Neurons

The experiments were carried out in male Sprague-Dawley rats (Harlan, Gannat, FRANCE) weighing 250 to 300 g at the day of the experiment and which have been kept under standard laboratory conditions (12:12 light-dark cycle with free access to food and water). The animals were anesthetized with chloral hydrate (400 mg/kg, i.p.). Supplemental doses were given to maintain constant anesthesia and to prevent any nociceptive reaction to a tail pinch.

Extracellular unitary recordings of dorsal raphe 5-HT neurons were performed with single-barreled glass micropipettes preloaded with fiberglass filaments in order to facilitate filling. The tip was broken back to 2 to 4 μm and filled with a 2M NaCl solution saturated with Blue Chicago dye. The rats (control or treated) were placed in a stereotaxic frame and a burr hole was drilled on the midline 1 mm anterior to lambda. Dorsal raphe 5-HT neurons were encountered over a distance of 1 mm starting immediately below the ventral border of the Sylvius aqueduct. These neurons were identified using the criteria of Aghajanian (Aghajanian, G. K., Essays Neurochem Neuropharmacol 2000, 3: 1-32): a slow (0.5-2.5 Hz) and regular firing rate and long-duration (0.8-1.2 ms) positive action potentials.

To determine putative synergistic effect of Compound II (2-(6-Fluoro-1H-indol-3-ylsulfanyl)-benzyl]-methyl-amine) on the inhibitory action of 5-HTP (25 mg/kg, i.v.) on the firing activity of dorsal raphe 5-HT neurons, a dose of Compound II corresponding to 20-30% of inhibition, was injected prior to and after the administration of 5-HTP.

Statistical analysis was performed with SigmaStat for Window version 4.0 software (Jandel Corporation). Average was the mean±SEM. Significance was considered for p<0.05.

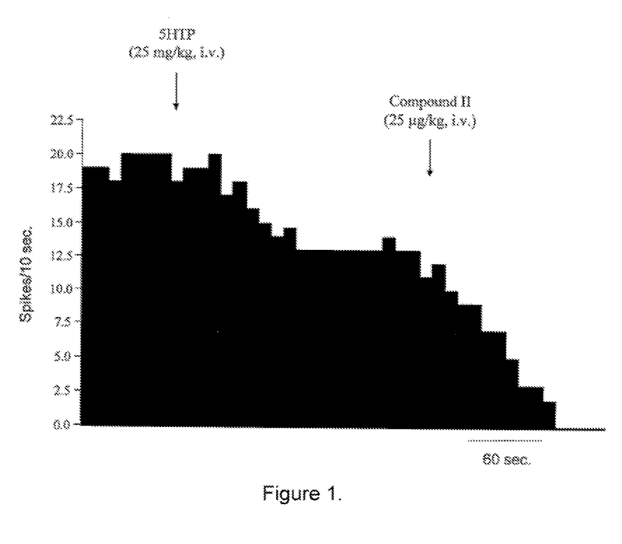

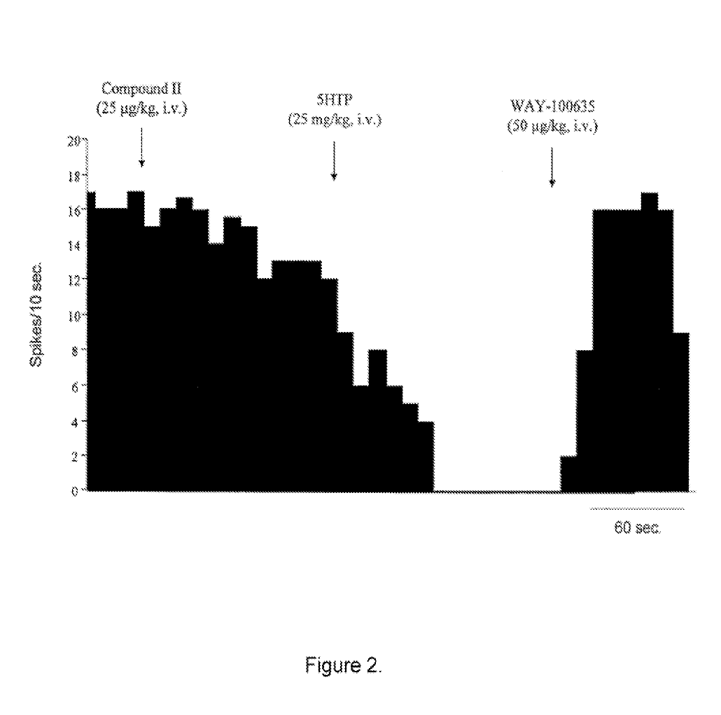

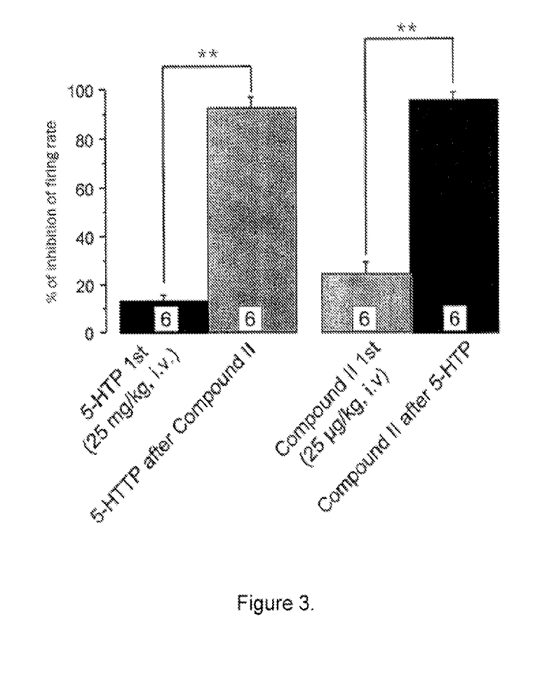

In a group of six rats, dorsal raphe 5-HT neurons had an average basal firing rate of 1.82±0.23 Hz. Injection of 25 mg/kg 5-HTP reduced the firing by 13.4±2.26% (see FIG. 1). These rats received Compound II (0.025 mg/kg) as the second injection and a further decrease of 82.43±2.78% was observed (FIG. 1). In another set of experiments, Compound II was injected prior to 5-HTP. In six rats with dorsal raphe 5-HT cell basal firing rate averaging 1.70±0.07 Hz, Compound II (0.025 mg/kg) inhibited the firing 24.1±4.98%, and subsequent injection of 5-HTP (25 mg/kg) further decreased the firing by 70.53±2.76% (FIG. 2). The degree of inhibition of dorsal raphe cell firing produced independently by each compound was: 5-HTP=13.4±2.26% and Compound II=24.10±4.98%. The combined effects for 5-HTP+ Compound II were markedly greater than that predicted by a simple additive interaction (additive=37.5%; 5-HTP first=95.83%; Compound II first=94.63%; FIG. 3). Hence, the effect of 5-HTP is significant greater in the presence of Compound II (p<0.01). Similarly, the effect of Compound II was found to be significantly greater in the presence of 5-HTP (p<0.01).

Subsequent administration of WAY-100635, which is a selective 5-HT_{1A} antagonist, increases the spontaneous firing rate of the neuron, suggesting that feedback inhibition of 5-HT neuronal firing has been decoupled by blocking 5-HT_{1A} receptors.

These results indicate that the combination of 5-HTP with Compound II produces effects on neuronal firing in the dorsal raphe nucleus that are synergistic and not additive.

### Inhibition of ³H-Serotonin Uptake in Rat Brain Synaptosomes

In order to test compounds at the primary, high-affinity binding site of the serotonin transporter, i.e. determine if a compound is a serotonin reuptake inhibitor, inhibition of the uptake of serotonin (5-HT) was determined.

By the following method, the inhibition of the uptake of ³H-serotonin (³H-5-HT) (10 nM) in rat brain synaptosomes by test compounds was determined in vitro. Exemplary methods, and also results of serotonin uptake for specific SRIs, is described in Hyttel, J., Psychopharmacology 1978, 60:13-18; Hyttel, J., Prog. Neuro-Psychopharmacol. & Biol. Psychait. 1982, 6:277-295; Hyttel, J. & Larsen, Acta Pharmacol. Tox. 1985, 56(suppl. 1):146-153; Sanchez, C. and Hyttel, J. European J. Pharm. 1994, 264:241-247; and Bøgesø, K., et al, U.S. Pat. No. 4,943,590, issued Jul. 24, 1990.

Procedure: Male Wistar (Mol:Wist) rats (125-250 g) were sacrificed by decapitation and exanguinated. Brain tissue (minus cerebellum) was gently homogenized (glass Teflon homogenizer) in 40 vol (w/v) of ice-cold 0.32M of sucrose containing 1 mM of nialamide. The P2 fraction (synaptosomal fraction) was obtained by centrifugation (600×g, 10 min and 25000×g, 55 min, 4° C.) and suspended in 800 volumes of a modified Krebs-Ringer-phosphate buffer, pH 7.4.

To 400 μl of the synaptosomal suspension (5 mg original tissue) on ice, 100 μl test compound in water was added. After preincubation at 37° C. for 5 min, 100 μl of ³H-5-HT (final concentration 10 nM) was added and the samples were incubated for 10 min at 37° C. The incubation was terminated by filtering the samples under vacuum through Whatman GF/F filters with a wash of 5 ml buffer containing 10 μM of unlabelled 5-HT. The filters were placed in counting vials and 4 ml of appropriate scintillation fluid (e.g. Picofluor™I5, Packard) was added. After shaking for 1 h and storage for 2 h in the dark, the content of radioactivity was determined by liquid scintillation counting (cpm). Uptake was obtained by subtracting the non-specific binding and passive transport measured in the presence of 10 μM test compound. The measured cpm was plotted against test compound concentration, and the best fitting s-shaped curve was drawn. The uptake inhibitory potencies are expressed as IC₅₀ values in nM (logarithmic means). Two full concentration-response curves were measured using five concentrations of test compound in triplicate. The IC₅₀ value was determined as the concentration at which the uptake is 50% of the total uptake in control samples minus the non-specific binding and uptake in the presence of 10 μM test compound.

2-(6-Fluoro-1H-indol-3-ylsulfanyl)-benzyl]-methyl-amine binds with high affinity to the serotonin transporter and exhibits uptake inhibitory potency (IC₅₀) of 0.35 nM.

### Allosteric Modulation of the Serotonin Transporter

The allosteric site of a protein is an additional binding site, which is distinct from the primary ligand binding site. Compounds that modulate, for instance increase and/or stabilize, binding between the ligand and the ligand binding site are generally considered to operate through an allosteric mechanism.

While not wishing to be bound by a particular theory, the serotonin transporter is considered to have at least two separate binding sites: a primary, high-affinity binding site that mediates the inhibition of serotonin reuptake, and one or more low-affinity binding sites that allosterically modulate the binding of ligands at the primary site (Plenge, P., and Mellerup, E. T. Eur J Pharmacol. 1985 Dec. 10; 119(1-2):1-8; Wennogle, L. P. and Meyerson, L. R. Life Sci. 1985 Apr. 22; 38(16):1541-50).

The binding of escitalopram to an allosteric binding site on the SERT has been demonstrated in several studies. Studies of the interaction of escitalopram with the human serotonin transporter expressed in COS-1 cell membranes demonstrated that escitalopram binds to a secondary low-affinity allosteric site and retards the dissociation rate of ³H-escitalopram (used in a concentration that exclusively binds to the high-affinity primary site) from the transporter; that is, escitalopram appears to have a stabilizing/self-potentiating effect on the escitalopram:serotonin transporter complex. The effect of escitalopram is concentration-dependent (See Chen, F., et al., Eur Neuropsychopharmacol. 2005 March; 15(2):193-8; and Chen, F., et al., J. Neurochem. 2005, 92:21-28).

In addition to escitalopram, the interaction of paroxetine, sertraline, fluoxetine, venlafaxine, duloxetine, and serotonin with high- and low-affinity binding sites on the human serotonin transporter expressed in COS-1 cell membranes has been investigated (Chen, F., et al., Eur Neuropsychopharmacol. 2005 March; 15(2):193-8). The study suggested that paroxetine, although to a lesser extent than escitalopram, stabilized the ³H-paroxetine:human serotonin transporter complex at the primary high-affinity site. Sertraline, fluoxetine, venlafaxine, and duloxetine had little or no stabilizing effect on their binding to the primary binding site on the serotonin transporter (Chen, F., et al., Eur Neuropsychopharmacol. 2005 March; 15(2):193-8).

Whether a compound operates through an allosteric mechanism can be determined by in vitro dissociation experiments. Dissociation binding experiments measure the “off rate” (k_{off}) for a radioligand of the protein. After radioligand and transporter protein are allowed to bind (i.e. form a complex), then ligand is added to block further binding of radioligand to the transporter so that the rate of dissociation can be measured. Binding (as measured by radioactivity of the radioligand:transporter complex) is measured at various times to determine the rate at which the radioligand dissociates from the transporter. Dissociation rate constants can be used to determine the half-life of the bound complex. Half-life determinations can be used to ascertain whether a compound is an allosteric modulator of the human SERT.

By performing dissociation binding studies, as similarly described in Chen, F C., J. Neurochem., 2005, 92, 21-28, it was determined that the compound 2-(6-Fluoro-1H-indol-3-ylsulfanyl)-benzyl]-methyl-amine binds to an allosteric site of SERT with an IC₅₀ of 26 uM and significantly slowed the dissociation rate of ³H-escitalopram.

In an alternative method, those of ordinary skill in the art can determine whether a compound, particularly an SRI, is an allosteric modulator of the human serotonin transporter (hSERT) as recited in the claims of this application, by determining the Z-factor for a compound by the method described in the following paragraphs.

To first determine the dissociation rate, isolated membranes from COS-1 cells transiently transfected with hSERT (GenBank Accession. No. X70697) are prepared by standard methods. Methods of transfection are also well known in the art. Hereinafter, assays are carried out in duplicate from at least three independent transfections using the same transfection method.

Initially, a radioligand/hSERT complex is formed during a 30-minute incubation of membrane preparations expressing hSERT and radioligand (radiolabeled-test compound) at 4° C. in buffer (50 mM Tris, pH 7.4; 120 mM NaCl, 5 mM KCl). Radioligand is present at a concentration approximately 10 times the K_{d} value for the radioligand. (K_{d} values are previously determined in the same buffer).

The radioligand/hSERT complex is diluted by 30-fold in the same buffer. In separate experiments the radioligand/hSERT complex is diluted by 30-fold in the same buffer containing test compound (cold, non-radiolabeled). Incubation of the radioligand/hSERT complex diluted in buffer with or without test compound continues for increasing time intervals at 20° C. At each time interval (e.g. 10 min., 20 min., 30 min., etc.), samples are removed from the incubation and the reaction is stopped by filtration through GF/C glass-fiber filters on a cell harvester. Accumulated radioactivity for each sample is determined by direct counting of plates using a Packard Bell microplate scintillation counter. The radioactivity represents binding and is expressed as fmol complex/mg membranes. Binding for each sample is plotted against increasing time to determine dissociation rate. The dissociation rate of the radioligand (k_{off}) is determined by non-linear regression using a GraphPad PRISM program (GraphPad Software, San Diego, Calif.). Dissociation half-life (t_{1/2}) is calculated by 0.69302/k_{off} and is represented in units of time.

Dissociation half-life of radioligand/hSERT complex (expressed in minutes) is plotted against increasing concentration of test compound in dissociation buffer (e.g. 10 μM, 20 μM, 30 μM, 40 μM, and 50 μM of test compound). The slope of this plot is termed a Z-factor. The Z-factor is calculated from at least four independent determinations. Z-factor is a measure of the degree of stabilization of the radioligand/hSERT complex. A Z-factor greater than 0 (zero) is indicative of a positive allosteric modulator. By way of non-limiting example to further illustrate the invention, R-citalopram does not fall within the class of SRIs, and is therefore not considered an allosteric SRI, because R-citalopram binds to the primary binding site of the serotonin transporter having a reported IC₅₀ value of greater than 50 nM. See, for example, Sanchez, C. et al. Psychopharmacology 2003; 167:353-362.

What is claimed:

1. A pharmaceutical composition comprising (i) 5-hydroxytryptophan in an amount ranging from about 1 mg to about 75 mg; and (ii) 2-(6-Fluoro-1H-indol-3-ylsulfanyl)-benzyl]-methyl-amine.

2. The composition of claim 1, wherein the composition comprises 1 mg to 50 mg 2-(6-Fluoro-1H-indol-3-ylsulfanyl)-benzyl]-methyl-amine.

3. The composition of claim 2, wherein the composition comprises 3 mg to 50 mg of 5-hydroxytryptophan.

4. The composition of claim 2, wherein the composition comprises 10 mg to 50 mg of 5-hydroxytryptophan.

5. The composition of claim 1, further comprising a peripheral decarboxylation inhibitor.

6. The composition of claim 5, wherein the peripheral decarboxylation inhibitor is carbidopa.

7. The composition of claim 6, wherein said composition contains carbidopa in an amount ranging from about 100 mg to about 150 mg.

8. The composition of claim 7, wherein said composition is a tablet or a capsule.

9. A pharmaceutical composition comprising (i) 5-hydroxytryptophan; and (ii) 2-(6-Fluoro-1H-indol-3-ylsulfanyl)-benzyl]-methyl-amine.

10. The composition of claim 9, wherein said composition contains 5-hydroxytryptophan in an amount ranging from about 1 mg to about 600 mg.

11. The composition of claim 9, wherein said composition contains 5-hydroxytryptophan in an amount ranging from about 25 mg to about 300 mg.

12. The composition of claim 9, wherein said composition contains 5-hydroxytryptophan in an amount ranging from about 50 mg to about 200 mg.

13. The composition of claim 9, wherein said composition comprises (i) 2-(6-Fluoro-1H-indol-3-ylsulfanyl)-benzyl]-methyl-amine in an amount from about 0.1 mg to about 50 mg and (ii) 5-hydroxytryptophan in an amount ranging from about 1 mg to about 600 mg.

14. The composition of claim 9, wherein said composition comprises (i) 2-(6-Fluoro-1H-indol-3-ylsulfanyl)-benzyl]-methyl-amine in an amount from about 0.1 mg to about 50 mg and (ii) 5-hydroxytryptophan in an amount ranging from about 25 mg to about 300 mg.

15. The composition of claim 9, wherein said composition comprises (i) 2-(6-Fluoro-1H-indol-3-ylsulfanyl)-benzyl]-methyl-amine in amount from about 0.1 mg to about 50 mg and (ii) 5-hydroxytryptophan in an amount ranging from about 50 mg to about 200 mg.

16. The composition of claim 9, further comprising a peripheral decarboxylation inhibitor.

17. The composition of claim 16, wherein the peripheral decarboxylation inhibitor is carbidopa.

18. The composition of claim 17, wherein said composition contains carbidopa in an amount ranging from about 100 mg to about 150 mg.

19. The composition of claim 18, wherein said composition is a tablet or a capsule.

20. The composition of claim 19, wherein said composition is in a unitary dosage form.
